(19) United States
(12) Patent Application Publication
(10) Pub. No.: US 2017/0245867 A1
(43) Pub. Date: Aug. 31, 2017

(54) DEVICE FOR THE PREPARATION OF A PATELLA FOR THE IMPLEMENTATION OF A PATELLAR IMPLANT

(71) Applicant: ASTON MEDICAL, Saint-Etienne Cedex (FR)

(72) Inventors: Bertrand Bouxin, Verton (FR); Didier Chabernaud, Flers (FR)

(21) Appl. No.: 15/521,591

(22) PCT Filed: Oct. 22, 2015
(86) PCT No.: PCT/FR2015/052846

§ 371 Date: Apr. 24, 2017

(30) Foreign Application Priority Data
Oct. 24, 2014 (FR) 1460248

(51) Int. Cl. A61B 17/15

(52) U.S. Cl. CPC A61B 17/158; A61B 2090/034

(57) ABSTRACT

The device comprises a clamp secured to a cutting guide (4) and comprising two arms (1) and (2) articulated against an elastic return means (3). According to the invention, the device comprises a bespoke volumetric body (5) acting as a spacer, being shaped such as to be coupled to one of the arms (2), one of the 10 faces of the body (5), considered to be opposite to the coupling with the arm (2) has a recessed indentation that is married, in inverted form, with the posterior articular face of the patella positioned on the other arm (1), said indentation and the height of the volumetric body (5) being defined by means of form modeling software using scanner or MRI data from said patella, in order to give the 15 orientation and positioning of the prosthetic patella, the posterior cutting height and the drilling depth of a retention pin for the patellar implant, the volumetric body (5) acting as a spacer being coaxially drilled with a through hole of a diameter that is substantially equal to that of the patellar implant retention pin.

The invention relates to the technical field of instruments, in particular orthopedic implants.

More particularly, the invention relates to a device for the customized positioning and cutting of a patella, and finds advantageous application in knee arthroplasty, and more particularly the resurfacing of the patella.

In a manner well known to those skilled in the art, a knee prosthesis, and more particularly the femoral implant included in this prosthesis, has, between condylar pads, a trochlea upon which the patella, during flexion of the lower limb, is capable of sliding. In order to avoid pain, the practitioner must, when implementing a prosthetic patellar implant, such as to avoid any shifting phenomena, respect the orientation of said implant with respect to the trochlea. The thickness should also be respected in order to avoid inappropriate tension of the patellar ligament on the trochlea of the prosthetic implant which is likely to lead to limited knee flexion.

Generally, the preparation of the anatomical patella, when implementing a prosthetic patellar implant, is performed by means of a clamp secured to a cutting guide. The design of this clamp does not however make it possible to prepare the patella for the specific pathological cases of patients to be fitted with a prosthesis.

The object of the invention is to remedy these drawbacks in a simple, safe, efficient and rational manner

The problem that the invention proposes to solve is to make it possible to prepare the anatomical patella by adapting, in particular, the positioning and height of the cut for the placement of the patellar implant, in a manner that is customized to the patella of the patient, in particular to the posterior face of the patella to be fitted as a prosthesis.

In order to solve such a problem, a device has been designed and developed for the preparation of a patella for implementing a patellar implant, comprising, in a known manner, a clamp secured to a cutting guide and comprising two arms that are articulated against an elastic return means.

According to the invention, the device comprises a bespoke volumetric body acting as a spacer, being shaped such as to be coupled to one of the arms, one of the faces of the body, considered to be opposite to the coupling with the arm has a recessed indentation that is married, in inverted form, with the anterior face of the patella positioned on the other arm, said indentation and the height of the volumetric body being defined by means of form modeling software using scanner or MRI data from said patella, in order to give the orientation and positioning of the prosthetic patella, the posterior cutting height and the drilling depth of a retention pin for the patellar implant, the volumetric body acting as a spacer being coaxially drilled with a through hole of a diameter that is substantially equal to that of the patellar implant retention pin.

These characteristics make it possible to give the orientation and the positioning of the prosthetic patella, the posterior cutting height and the drilling depth of the prosthetic patellar implant retention pin.

In order to solve the problem of coupling the volumetric body to one of the arms of the clamp, in particular the upper arm, the hole of the volumetric body is extended by means of a centering pin that is capable of cooperating with a complementary shape of one of the arms of the clamp, in particular the upper arm, said body having a planar support surface cooperating with said arm.

In order to solve the problem of determining the correct drilling depth of the anatomical patella, the arm, whereupon the volumetric body is coupled, serves as an abutment for a drill-bit engaged in the hole of the volumetric body. Said abutment is located at the drilling depth of the patella taking into account the adapted height of said body.

In one embodiment, the two arms of the device each receive at their free end, an articulated arm, said arms receiving the volumetric body and being mounted with height-displacement capability in order to be spaced apart or brought closer together, in a parallel manner, in combination with a guide column retaining the return means.

In order to solve the problem of positioning the anatomical patella on one of the arms of the clamp in order to position the volumetric body for the preparation of said patella, the end of the arm relative to the lower arm has rough anchoring patches that cooperate with the patella.

To resolve the problem of implementing the cutting of the front of the patella, the cutting device guide is mounted with height adjustment on the guide column.

The invention is described below in more detail using the figures of the drawings attached, wherein:

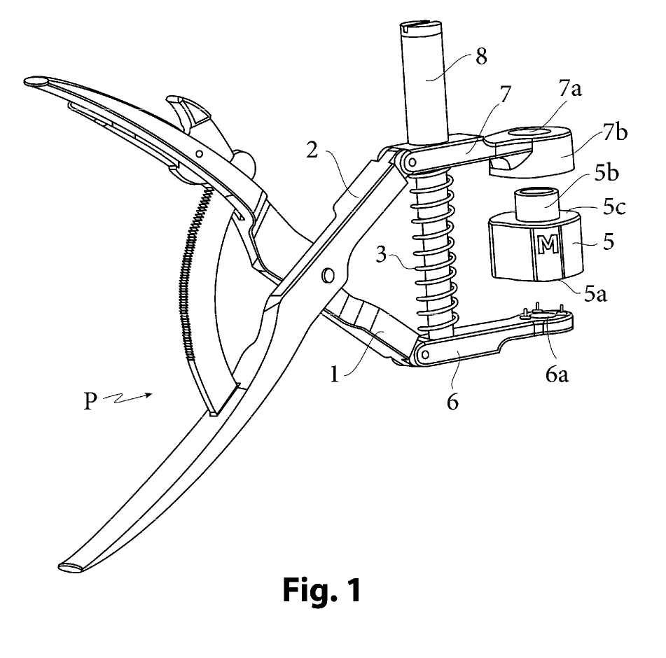

FIG. 1 is a perspective view of the device, before the implementation of the spacer in relation to the anatomical patella,

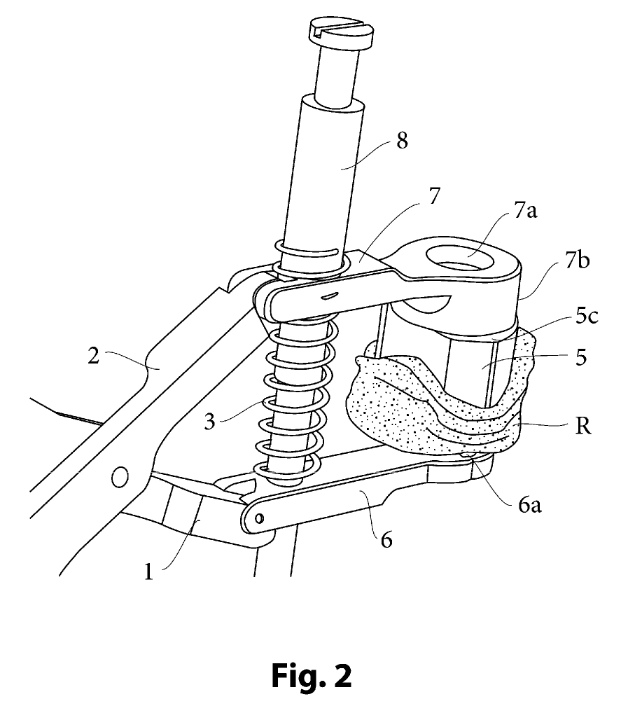

FIG. 2 shows the device for the preparation of the patella positioned in relation to the spacer, after tightening the arms of the clamp,

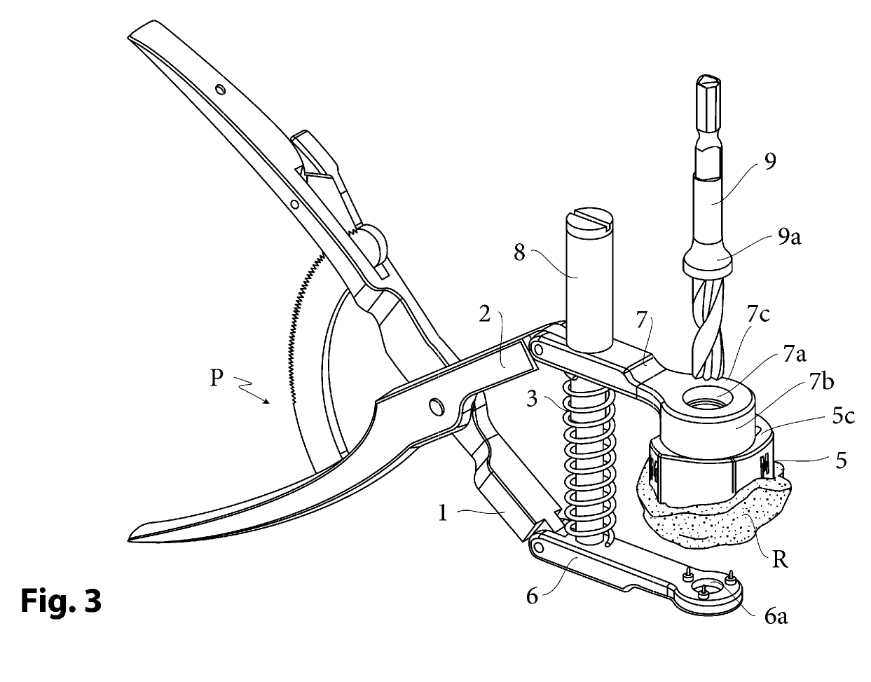

FIG. 3 shows the device before the drilling of the indentation of the patellar pin with a drill-bit.

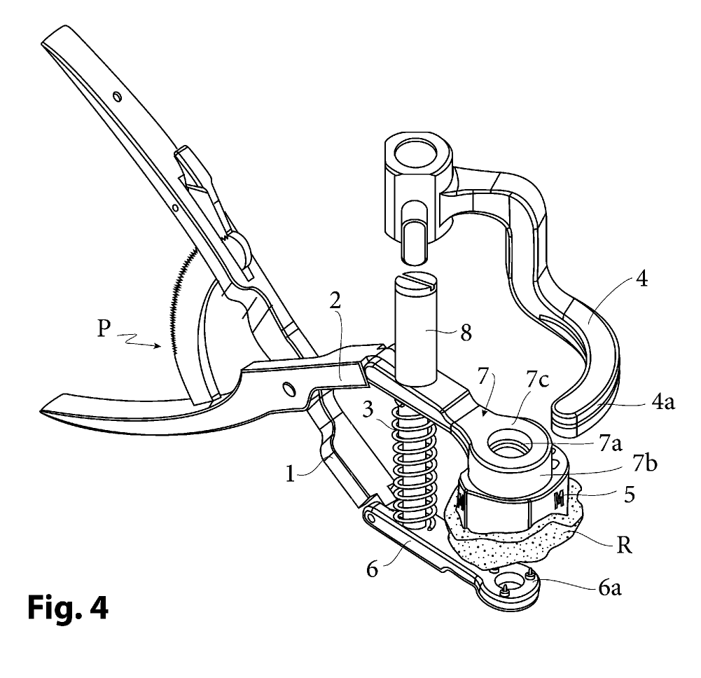

FIG. 4 is a perspective view of the device, before the implementation of the spacer in relation to the anatomical patella,

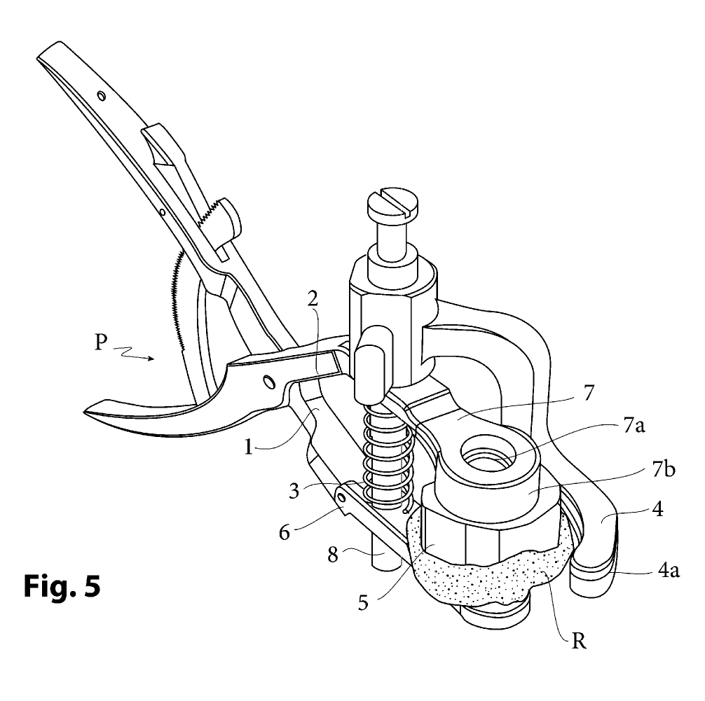

FIG. 5 is a perspective view of the device, in the clamped arms position with the spacer and the anatomical patella, the cutting guide being blocked in the lower position on the face of the upper arm, and determined by the correct height of the spacer in order to ensure the cutting of the front of the anatomical patella, by means of a cutting member, such as a saw.

The device, according to the invention, for the development of a patella, comprises a clamp designated in the entirety thereof by (P) and having two arms (1) and (2), articulated against an elastic return means (3), particularly in the form of a spring. Said clamp (P) is, moreover, secured to a cutting guide (4).

According to a characteristic of the invention, the clamp includes a bespoke volumetric body (5), of a generally parallelepiped form, and acting as a spacer. The spacer (5) is intended to be temporarily connected to one of the arms on the clamp, particularly the upper arm (2), as will be indicated later.

Importantly, one of the faces (5a) of the body (5), that is considered to be opposite the face that couples with the arm (2), has a recessed indentation, that marries, in inverted form, with the front of the patient's anatomical patella being positioned on the other arm (1). Said indentation and the height of the volumetric body (5) are defined by means of form modeling software using scanner or MRI data from the anatomical patella. These provisions make it possible to give the orientation and the positioning of the prosthetic patella, the posterior cutting height and the drilling depth of the patellar implant retention pin.

In the illustrated embodiment, the two arms (1) and (2) each receive an articulated arm (6) and (7) at their free ends. The arms (6) and (7) are mounted mounted with height-displacement capability in order to be spaced apart or brought closer together, in a parallel manner, in combination with a guide column (8) under the effect of activating the arms (1) and (2) of the clamp. The guide column (8) retains the return spring (3) mounted between the two arms (6) and (7).

The volumetric body (5) is coaxially drilled with a through-hole of a diameter that is substantially equal to that of the prosthetic patellar implant retention pin. Said hole is extended by a centering pin (5b) that is able to cooperate with a complementary indentation (7a) that has a circular journal (7b), formed at the free end of the arm (7) of the upper arm (2). After the pin (5b) engages in the indentation (7a) of the journal (7b), a flat support face (5c) of the spacer (5), cooperates with said journal (7b).

The arm (7), whereupon the volumetric body (5) is coupled, serves as an abutment for a drill (9) engaged in the hole aligned with the indentation (7a) of the journal (7b), of the pin (5b) and of the body (5). In the example illustrated, as shown in FIG. 3, the body of the drill (9) has an abutment (9a) that can be supported on the upper face of the arm (7) at the level of the face (7c) of the circular journal (7b).

The free end of the lower arm (6) has rough anchoring patches that cooperate with the anatomical patella, as shown in FIG. 2.

It is apparent from these characteristics that the spacer (5), with respect to the support surface that marries, in inverted form, with the internal face of the anatomical patella, the shape thereof being defined by form modeling software using scanner or MRI data that the practitioner will have asked of his patient, allows the positioning and orientation of the clamp to be defined with respect to the posterior articular face of the patella.

As indicated, FIG. 3 shows the preparation of the patella (R) positioned with regard to the volumetric body (5), before the drilling of the hole for the patellar pin, with the drill (9), positioned within the hole (7a) and butted against the journal (9a) on the face (7c) of the aim (7). This abutment determines, by means of the height of the volumetric form (5), the correct drilling of the anatomical patella.

FIG. 4 shows the preparation of the anatomical patella (R) secured to the spacer (5), before implementing the cutting guide (4) on the column (8) and in FIG. 5, the cutting guide (4) is implemented on the column (8) and is locked in the lower position on the upper face of the arm (7), for example by means of a wheel. The cutting guide (4) is positioned at the correct cutting height taking into consideration the correct height of the volumetric body (5), the height thereof being bespoke, in relation to scanner or MRI data, therefore allowing for the cutting of the posterior articular face of the anatomical patella by means, for example, of a saw blade inserted into a slot (4A) within the cutting guide (4). The prosthetic patella finds itself, therefore, in the desired orientation.

The advantages are apparent from the description.

1. Device for the preparation of a patella for the implementation of a patellar implant, comprising a clamp secured to a cutting guide (4) and comprising 2 aims (1) and (2) articulated against an elastic return means (3), characterized in that it comprises a bespoke volumetric body (5) acting as a spacer, being shaped such as to be coupled to one of the aims (2), one of the faces of the body (5), considered to be opposite to the coupling with the aim (2) has a recessed indentation that is married, in inverted form, with the posterior articular face of the patella positioned on the other arm (1), said indentation and the height of the volumetric body (5) being defined by means of form modeling software using scanner or MRI data from said patella, in order to give the orientation and positioning of the prosthetic patella, the posterior cutting height and the drilling depth of a retention pin for the patellar implant, the volumetric body (5) acting as a spacer being coaxially drilled with a through hole of a diameter that is substantially equal to that of the patellar implant retention pin.

2. Device according to claim 1, characterized in that the hole of the volumetric body (5) is extended by means of a centering pin (5b) that is capable of cooperating with a complementary shape of one of the arms (2) of the clamp, in particular the upper arm, said body (5) having a planar support surface (5c) cooperating with said arm (2).

3. Device according to claim 2, characterized in that the arm (2) whereupon the volumetric body (5) is coupled, serves as an abutment for a drill (9) engaged in the hole of the volumetric body (5), said abutment being located at the drilling depth of the patellar taking into consideration the adapted height of said body.

4. Device according to claim 1, characterized in that the two arms (1) and (2) each receive at their free end, an articulated arm (6) and (7), said arms receiving the volumetric body (5) and being mounted with height-displacement capability in order to be spaced apart or brought closer together, in a parallel manner, in combination with a guide column (8) receiving the return means (3).

5. Device according to any of claims 1 to 4, characterized in that the end of the arm (6) relative to the lower arm (1), has rough anchoring patches that cooperate with the patella.

6. Device according to claims 1 and 4, in that the cutting guide (4) is mounted with height adjustment on the guide column.
